(19) United States
(12) Patent Application Publication
(10) Pub. No.: US 2006/0004398 A1
(43) Pub. Date: Jan. 5, 2006

(54) Sequential dilator system

(76) Inventors: Lawrence J. Binder JR., Doylestown, PA (US); David Gerber, West Chester, PA (US)

Correspondence Address: JONES DAY, 222 EAST 41ST ST, NEW YORK, NY, 10017, US

(21) Appl. No.: 10/884,705

(22) Filed: Jul. 2, 2004

(51) Int. Cl. A61M 29/00

(52) U.S. Cl. 606/191

(57) ABSTRACT

The present invention is directed to a sequential dilator for use in surgery and a method for using the sequential dilator. The sequential dilator may have a bullet-shaped dilator and a plurality of dilator tubes with a removable handle. The method may include inserting a guide wire through an incision into a patient's vertebra and subsequently inserting the bullet-shaped dilator and dilator tubes with tapered ends and of increasing size into the incision to increase the size of the incision. A kit including the components necessary for the method is also disclosed.

### FIELD OF THE INVENTION

The present invention relates generally to a sequential dilator system, and more particularly to a sequential dilator system for use in surgery in creating access openings to the posterior spine for discectomy, interbody fusion, and pedicle screw fixation.

### BACKGROUND OF THE INVENTION

The subject disclosure relates to minimally invasive surgical procedures and apparatus, and more particularly to a system for sequentially dilating an incision for performing minimally invasive surgery on the spine. A variety of retractors and dilation systems have been used to provide a traditional “open-incision” approach to the posterior spine, as well as for providing the more modern “minimally invasive” access to the spine. Problems associated with the surgical instruments and systems commonly used to provide such an “open incision” include the size of the instruments, which may be large and may occupy a significant portion of the surgical space to allow the surgeon a sufficiently large field in which to work. Sequential dilation systems provide an advantage in that they allow the surgeon to make an initially small incision, then gradually increase the size of the opening to the minimum size required for performing the surgical procedure, thus reducing tissue damage and speeding recovery patient time. The current invention provides a sequential dilator system that may be used to establish a minimally invasive opening through which surgical procedures may be performed on the spine or other areas of the body, and which is easy to install, manipulate and remove.

### SUMMARY OF THE INVENTION

While the description of the dilator of the present invention relates to a sequential dilator system used in orthopedic surgery procedures, it should be understood that the retractor will find use not only in orthopedic surgery, but in other surgical procedures in which a surgeon wishes to gain access to an internal cavity by cutting the skin and going through the body wall in order to keep the incision spread apart so that surgical instruments can be inserted.

The dilator may comprise a handle suitable for grasping by a user and a series of dilator tubes of increasing diameter and shorter lengths, all with a tapered end for insertion into a patient. Preferably, the handle contains two or more sections of different internal diameter, each section including a dilator tube retaining mechanism. The different diameter sections of the handle match the outside diameters of the dilator tubes, which also include handle-engaging surfaces on the end opposite that inserted into the patient to mate with the dilator tube retaining mechanisms of the handle. There may be multiple grooves or other handle-engaging surfaces in the end of the dilator tubes that can be used to assist in grasping the dilator tubes and/or for use in a color coding system to indicate lengths, diameters, materials, etc. The handle may also have a window that allows the surgeon to determine when a dilator tube has engaged one of the ball detents of the handle. The internal diameters of the handle and outer diameters of the dilator tubes may further have matching flats to prevent relative rotation between the handle and dilator tubes.

An incision is made over the surgical site and a guide wire is driven through the tissue using a trocar. The guide wire is then inserted into bone using a mallet. The smallest of a series of dilator tubes is slipped over the end of a bullet-shaped dilator. The trocar is removed and a bullet-shaped dilator is guided over the wire and pressed down into the incision. The dilator tube that was slipped over the bullet-shaped dilator is then inserted into the incision over the bullet-shaped dilator, widening the incision, and the bullet-shaped dilator is removed. The next larger dilator tube is inserted into the handle such that it engages a ball detent. The assembly of handle and dilator tube is then placed over the smallest dilator tube and pressed down through the incision, widening the incision. When the assembly of handle and dilator tube is inserted, the dilator tube already in the patient will engage a ball detent. The surgeon may then grasp the outer dilator tube and remove the assembly of handle and inner dilator tube. The handle is then removed from the second dilator element and is fit over the next larger size dilator element, which is then pressed down into the incision over the dilator tube in the incision, further widening the incision. This procedure is repeated using larger and larger dilator elements until the desired incision size is obtained.

When the desired incision size is obtained, a working cannula may be inserted through which a surgical procedure may be conducted. The working cannula may be attached to a rigid frame, to which other working cannulae may be attached.

The materials and equipment necessary for carrying out the method of the invention may be presented for use in the form of a kit. The kit may include a guide wire, a T-shaped trocar, a mallet, a bullet-shaped dilator, dilator tubes, a handle or handles, and working cannulae. The components of the sequential dilator may be made from any combination of metals (such as, but not limited to, stainless steel or aluminum), composites (such as, but not limited to, carbon fiber composite), and polymers (such as, but not limited to, polyether ketone (PEEK) or ultra high molecular weight polyethylene (UHMWPE)). It may be desirable to make the working cannulae from a radiolucent material such as polyetherether ketone (PEEK).

### BRIEF DESCRIPTION OF THE DRAWINGS

While preferred features of the present invention are disclosed in the accompanying drawings, the invention is not limited to such preferred features wherein:

FIG. 1 is a perspective view of the placement of a guide wire into a surgical incision;

FIG. 2 is a side view of a bullet-shaped dilator;

FIG. 3 is a perspective view of the insertion of the bullet-shaped dilator of FIG. 2 into the surgical incision of FIG. 1;

FIG. 4 is a side view of a dilator tube handle;

FIG. 5 is an end view of the dilator tube handle of FIG. 4;

FIG. 6 is a section view of the dilator tube handle of FIG. 4;

FIG. 7A is a section view of a dilator tube;

FIG. 7B is a cross-sectional view of the dilator tube of FIG. 7A along A-A;

FIG. 8 is a side/section view of a working cannula;

FIG. 9 is an end view of the working cannula of FIG. 8; and

FIG. 10 is a view of a series of six individual dilator tubes that make up a sequential dilator set.

### DETAILED DESCRIPTION OF THE PREFERRED EMBODIMENTS

While the description of the dilator system of this invention will be discussed primarily in relation to spinal surgery, it should be understood that the system will find use in other areas of surgery in which a surgeon wishes to gain access to an internal cavity by cutting the skin and enlarging an incision in a body wall so that surgical instruments can be inserted to perform a desired surgical procedure. For example, the dilator system may be used to create an incision 100 to provide access to the posterior spine through which pedicle screws may be percutaneously installed in one or more selected vertebra. Alternatively, the dilator system may be used to create an incision to access an intervertebral disc space for performance of a minimally invasive discectomy procedure and/or spinal fusion procedure, including the implantation of one or more intervertebral implants.

As shown in the accompanying figures, the dilator system may comprise a bullet-shaped dilator instrument 400, one or more dilator tubes 600, at least one removable handle 500 suitable for manipulating and inserting the one or more dilator tubes, and at least one working cannula 1200. All elements may also be cannulated so that they may be guided to the surgical site using a pre-installed guide wire 200. Where more than one dilator tube is provided, each tube in the series may comprise a slightly larger diameter in comparison to the previous tube in the series, thus when they are inserted into the incision 100 one after another, they may facilitate a gradual, sequential, expansion of a surgical incision, thus reducing the likelihood for damaging surrounding tissue. The bullet-shaped dilator instrument 400 may be inserted into the incision 100 and used to form the primary opening to the surgical site. After the bullet-shaped dilator 400 is fully inserted, the individual dilator tubes 600, 700 may then be inserted, one after another, to sequentially expand the incision to the size desired for the desired procedure. The dilator tubes 600, 700 may each be provided with a tapered insertion end 610, 710 configured to facilitate insertion of the tubes in the surgical incision 100. The dilator tubes also may have an opposite end comprising surface features 622, 722 configured to engage the removable handle 500 and/or to allow the user to grip the tubes by hand.

The handle 500 may be configured to engage at least one dilator tube 600 to enable the surgeon to more easily manipulate the tube within the incision. Once used to insert the tube 600 into the incision, the handle may be removed from the tube, thus allowing access to the surgical site via the tube. Where more than one dilator element will be used, the handle may then be attached to the next larger dilator tube 700 and used to insert that tube over the previous tube 600 to incrementally expand the incision 100. Advantageously, the handle 500 may have a feature that allows it simultaneously engage the smaller tube 600 upon insertion of the larger tube 700 in the incision. Thus the smaller tube 600 may be conveniently removed from the incision 100 while the larger tube 700 is left in place. This process may be repeated using larger dilator tubes 800, 900 until the incision has been expanded to the desired size. Thereafter, the working cannula 1200 may be inserted and the surgical procedure may be performed through the cannula.

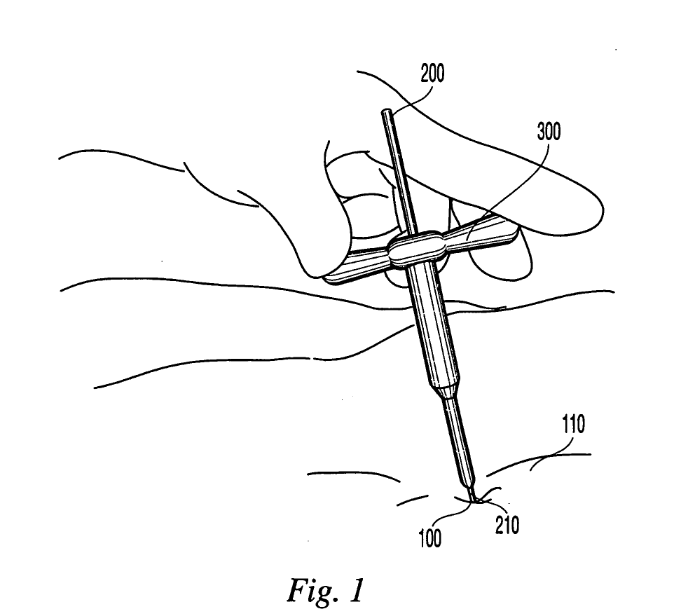

Referring to FIG. 1, an entry point may be selected on the patient's skin to obtain access to the targeted surgical site, and an incision 100 of appropriate length may be made through the dermal layers 110 of a patient's body at the entry point. The tip 210 of a guide wire 200 may then be positioned within the incision 100 and guided toward the spine using a cannulated T-handled trocar 300. Once the tip 210 of the guide wire 200 penetrates the tissue overlaying the spine and contacts the pedicle of the targeted vertebra, the guide wire 200 may be driven into the pedicle using a mallet. Trocar 300 may then be removed from guide wire 200, leaving one end of the guide wire engaged with the pedicle and the opposite end extending out of the patient's body through the incision. The guide wire 200 may then be used to easily and accurately guide the successive dilator elements to the surgical site. For a discectomy, the guide 200 wire may be driven either adjacent to or directly into the disc rather than the vertebral pedicle. For surgical procedures performed on parts of the body other than the spine, the guide wire 200 may be driven into another bone or even another body part. The dilator system may also be used without a guide wire 200, in which case the surgeon may place the elements guided by fluoroscopy or other imaging or navigation techniques.

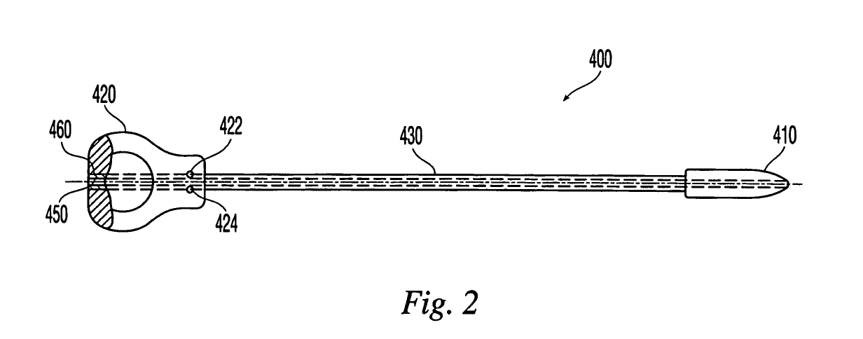

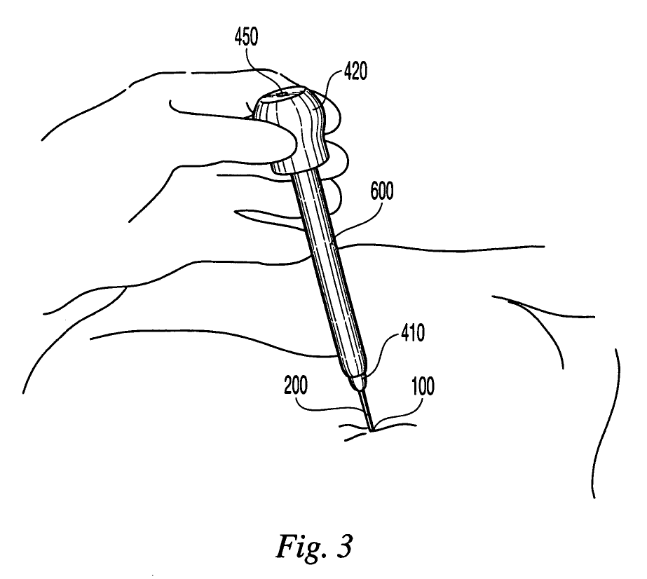

Referring to FIG. 2, bullet shaped dilator 400 may have an enlarged distal end 410 with a roughly parabolically tapered leading end surface, and a proximal handle end 460 to which a handle 420 may be fitted. An intermediate shaft 430 may extend between the proximal and distal ends, and may attach to the handle 420 using pins 422 and 424. Enlarged distal end 410 may be made as a single piece integral with shaft 430 or it may be attached to the shaft 430 by welding, brazing, threads or other appropriate means well-known in the art. The handle 420 may also be attached to shaft 430 by welding, brazing, threads, or other means well-known in the art. The bullet-shaped dilator 400 may have a central cannulation configured to slidingly engage a surgical guide wire 200, thus, the dilator may be guided down to the surgical site via the guide wire 200 which, as previously noted, may be placed in the targeted vertebra in a prior procedural step. FIG. 3 shows the enlarged distal end 410 of bullet-shaped dilator 400 positioned over guide wire 200, ready to be driven down through the tissue to initially expand the incision 100.

After the bullet shaped dilator 400 has been fully inserted into the incision 100 such that its distal end 410 lies adjacent the surgical site, it may then be removed by pulling it back up along the guide wire. It may also be left in place to serve as a guide for the first sequential dilator element 600. For procedures in which the bullet shaped dilator 400 is immediately removed, the smallest of the sequential dilator elements 600 may thereafter be introduced directly over the guide wire 200 and into the incision. For procedures in which the bullet shaped dilator 400 is left in place to serve as a subsequent guide, sequential dilator element 600 may be introduced directly over the bullet-shaped dilator. When the latter procedure is used, the handle 420 of the dilator 400 may be removed prior to inserting sequential dilator element 600. Alternatively, the bullet shaped dilator may be provided with an integral handle (not shown) having a diameter smaller than the inner diameter of the first sequential dilator element 600 so that the handle needn't be removed to allow the dilator element to be inserted into the incision.

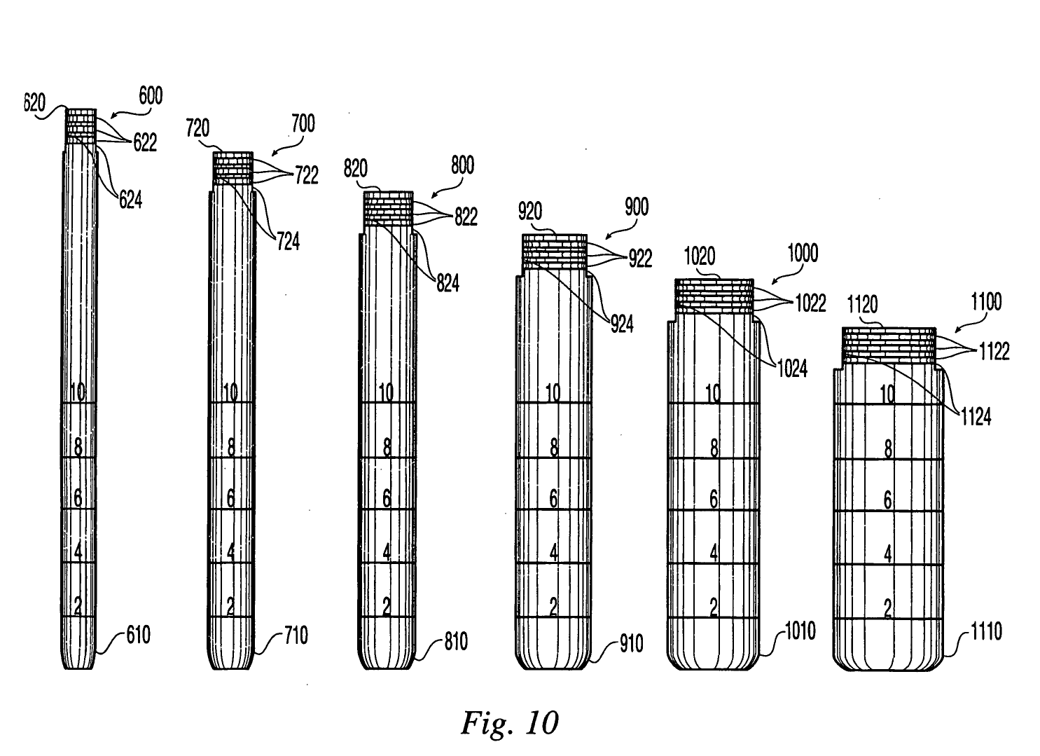

In an alternative embodiment, the smallest of the series of dilator tubes 600 may be placed over the bullet-shaped dilator 400 prior to insertion of the bullet-shaped dilator in the patient. It should be noted, however, that any number of dilator tubes, for example, dilator tubes 600, 700, 800, 900, or 1000 (FIG. 10) may be placed over the bullet-shaped dilator 400 prior to insertion of the bullet-shaped dilator 400 into the patient. In this embodiment, the initial dilation step may amount to a greater initial expansion of the incision as compared the case in which only the bullet-shaped dilator is used. And upon removal of the bullet-shaped dilator 400 from the patient, the smallest dilator tube 600 (or any number of dilator tubes) may remain in the patient.

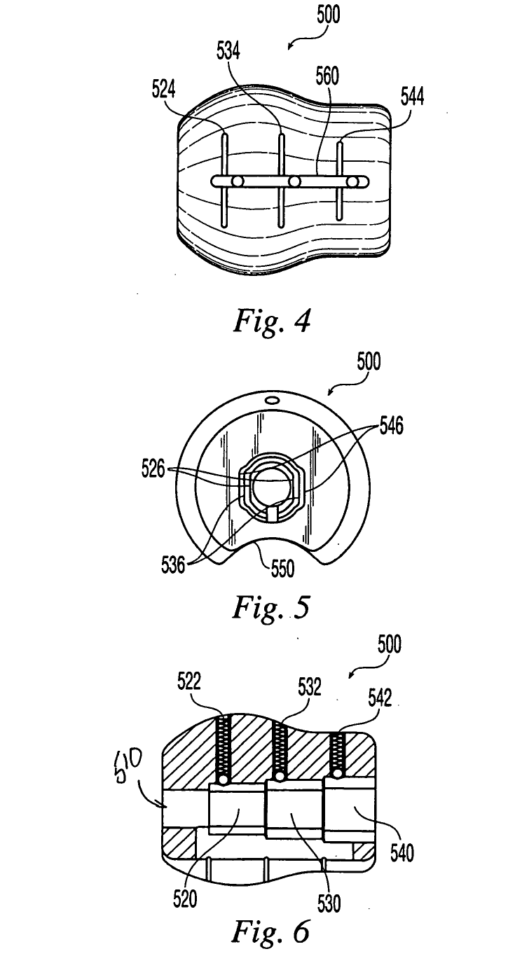

Referring to FIGS. 4-6, handle 500 may be ergonomically shaped and have a through hole 510, the through hole having counterbores 520, 530, and 540 of increasing diameter, set at different heights “h1,” “h2,” “h3,” within the handle, and sized to slidingly receive dilator tubes of successively larger diameter. Although the through hole 510 is shown, it is not required and handle 500 may be constructed without a through hole. When a guide wire (e.g., guide wire 200) is used in conjunction with a handle 500 to guide dilators into a patient, handle 500 preferably has some portion, such as through hole 510, that can provide for the passage of the guide wire 200 through the handle 500. Counterbores 520, 530, and 540 each may have a dilator tube-retaining mechanism configured to coact with corresponding surface features on the associated dilator tubes to retain the dilator tubes axially with respect to the handle. In the illustrated embodiment, the tube retaining mechanisms comprise ball detent mechanisms 522, 532, and 542 associated with counterbores 520, 530, and 540, respectively, and which are configured to engage a corresponding circumferential grooves 622, 722, 822 in dilator tubes 600, 700, 800, respectively. The ball detent mechanisms 522, 532, 542 may be commercially available assemblies that can be inserted into threaded holes in handle 500. The ball detent mechanism further may be configured to release an engaged dilator tube when a specified axial pressure is applied to the dilator tube, thus allowing the dilator tube to be separated from the handle by hand.

In an alternative embodiment, the dilator tube retaining mechanism may be provided as a spring-loaded button that may allow release of an engaged dilator tube simply by pressing or pulling on the button. Such a retaining mechanism may reduce the amount of force that must be applied to the tube to grip the outer diameter to hold the tube stationary while the handle is pulled out and away from the handle.

In a further alternative embodiment, each counterbore 520, 530, 540 may comprise at least one raised projection (not shown) configured to engage a respective dilator tube circumferential groove. The projection may be partially or completely rigid, so that during insertion of the tube in the handle counterbore, the projection may cause the proximal most portion of the dilator tube to undergo a slight elastic deformation, thus allowing the projection to slip into the appropriate groove. When seated in the appropriate groove, the projection would provisionally retain the tube within the handle. Removal of the tube from the handle would again cause the tube proximal end to flex inward slightly as the projection is slipped out of the groove. The projection may be in the form of a circumferential ridge, which may extend about at least a portion of the inner circumference of the counterbore. The projection may be in the form of at least one raised bump, or a set of discrete raised bumps which may be configured to engage a respective dilator tube groove. In yet another embodiment, the dilator tube proximal end may comprise at least one projection, and the handle counterbore may comprise a corresponding recess configured to engage the projection. Further examples of other connection schemes for retention of a dilator tube on a handle are corresponding tapered surfaces, corresponding threaded surfaces, corresponding toothed surfaces, etc. Alternatively, the handle may be provided as two half portions connected by a hinge, such that a dilator tube may be engaged/disengaged with the handle by closing/separating the handle halves. It will thus be appropriated that any appropriate retention mechanism may be provided, as long as it allows for easy engagement and disengagement of the handle and dilator tube by the surgeon.

Recessed portion 550 of handle 500 may further include a viewing window 560 to allow the surgeon to view the position of a dilator tube as it is being inserted into, or removed from, the handle 500. Further, the handle 500 may have one or more visual depth markings 524, 534, 544 located adjacent the viewing window 560 to allow the surgeon to visually determine when the end of an associated dilator tube has been fully inserted into handle 500 such that it engages an associated ball detent 522, 532, 542. These markings may comprises grooves, etchings, or any other appropriate marking. Thus, at least a portion of the proximal end of a dilator tube may be visible through the window when the dilator tube is engaged with the associated ball detent.

In an alternative embodiment, a proximal portion of one or more dilator tubes may have one or more viewing windows to allow the surgeon to determine the relative position of a smaller dilator tube within the larger tube. Thus, when a larger dilator tube is inserted over a smaller tube (either during installation of the larger tube or removal of the smaller tube), the surgeon may view the relative position of the smaller tube within the larger tube through the window.

In the embodiment of the dilator handle 500 having ball-detent retention mechanisms, the shape of the grooves of the dilator tubes may be configured to enhance the audible click or tactile “feel” of the ball engaging the groove to provide the surgeon with an appropriate non-visual feedback that indicates the tube is adequately engaged with the handle.

As shown in FIG. 5, counterbores 520, 530, 540 may have at least one flattened side 526, 536, 546 configured to engage a corresponding flattened side of each tubular dilator tube to prevent relative rotation between handle 500 and the dilator tubes. This may be advantageous during insertion of the dilator element in the patient as it allows the dilator tube to be twisted using the handle. Such twisting may aid or ease the movement of the dilator element down into the surgical incision by overcoming frictional forces or the forces of soft tissue that may tend to adhere to the outside of the dilator tube. It is noted that while the illustrated embodiment shows corresponding flattened sides, any other appropriate arrangement known in the art may be used to rotationally lock the handle to the dilator tube. Thus, corresponding axial grooves and protrusions may be provided in the corresponding surfaces of the handle counterbores and the dilator tubes. Likewise, the corresponding surfaces of the counterbores and tubes may be provided as geometric shapes, such as square, hexagonal, etc. Still other known rotational locking arrangements may also be used for this purpose.

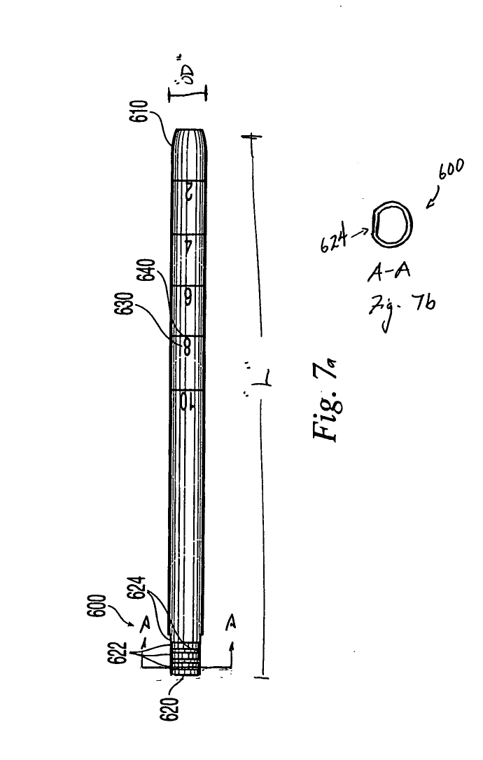

Referring to FIG. 7A, dilator tube 600 may have a tapered distal end 610 configured for insertion into the incision and a proximal end 620 configured to be grasped by the user for manipulation of the dilator tube. The tapered distal end 610 may comprise any configuration appropriate to provide a smooth expansion of patient tissue when the dilator tube 600 is inserted into an incision in the patient. Thus, the distal end 610 may comprise a straight taper having an appropriate taper angle, or it may comprise a curved taper of any appropriate geometry (e.g. parabolic, compound). The distal end 610 may also comprise any combination of straight and curved tapers, and different sequential dilator elements may comprise different taper configurations and geometries. In the illustrated embodiment, the taper of distal end 610 spans approximately 10 millimeters (mm) from the distal end of the tube 600 and progresses at a radius of about 50 mm, ending in a rounded distal end of about 0.1 mm radius, which may also be the approximate thickness of the dilator tube at the distal end. Other taper dimensions may be used to provide the desired smooth installation of the tubes into the incision, as will be apparent to one of skill in the art.

As previously described, the grooves 622 in dilator tube 600 may serve multiple purposes, such as allowing a user to manually grasp the proximal end to manipulate the tube during surgery, and/or facilitating engagement of the tube with the handle 500. The grooves may also be colored, and the grooves of different sized dilator elements may have different colors, where each color may signify the particular diameter, length, material, etc. of a particular dilator tube so as to make identification of tubes easier for the user. Handle 600 may also be color coded to ensure it is used with dilator tubes of the proper diameter.

In a further embodiment, the proximal end 620 of dilator tube 600 may comprise at least one flattened side 624 (FIGS. 7A and 7B) and preferably two flattened sides 624 configured to mate with a corresponding flattened side 526 of a respective counterbore 520 of handle 500. As explained above, flattened sides 526 and 624 may prevent rotation of the tube with respect to the handle 500, thus allowing the assembled dilator tube 600 and handle 500 to be twisted upon insertion of the tube in the incision 100. As further explained above, the mating portions of handle 500 and dilator tube 600 may be provided with other means of preventing rotation such as corresponding axial grooves and protrusions. It is also noted that using dilator tubes without such flats may provide the advantage in that it allows the user to engage the handle with a tube without requiring the user to align the respective flats of the tube and handle.

Numbers 630 and/or line markings 640 also may be provided on the outer surface of dilator tube 600 to allow the surgeon to determine the length that the dilator tube 600 has been inserted into the patient, thus allowing the surgeon to select the length of the working cannula that will ultimately be used. Such numbers and lines may be provided by etching, printing, stamping or any other appropriate method known in the art.

As can be seen in FIG. 10, dilator tube 600 may be the smallest of a series of dilator tubes in which each successively larger dilator element has an increased diameter as compared to the previous dilator element. Likewise, each successively larger dilator element may have a shorter length than the previous element, thus allowing easy user-access to the proximal end of the previous dilator element for removal once the next larger element has been placed in the patient. Each dilator tube should be of sufficient length so that at least a portion of each tube (i.e. the portion of the proximal ends comprising the gripping surface) extends outside of the patient when the distal end of the tube is positioned within the patient and adjacent the surgical site. In one embodiment, dilator tube 600 may have an outer diameter “OD” of about 12.7 mm and a length “L” of about 210 mm. Subsequently larger dilator tubes may be about 15 mm shorter in length, and 2-3 mm larger in diameter as compared to the previous tube in the sequence. It is noted, however, that any appropriate incremental changes in length and width may be used to suit the surgical circumstances, as will be apparent to one of skill in the art.

The clearance between the outside diameter of one dilator tube and the inside diameter of the next successive dilator tube should be sufficient to allow for easy installation of a next larger dilator tube and to avoid binding between the tubes, but should not so large as to allow tissue to be caught or pinched between the tubes during installation. In one embodiment this clearance may be from about 0.4 mm to about 0.7 mm. Further, although the dilator tubes are shown as cylindrical, dilator tubes may be provided in any appropriate cross-sectional shape, including but not limited to, oval, elliptical, figure-eight, etc.

The handles, bullet-shaped dilator, and dilator tubes of the sequential dilator may be made from any combination of metals (such as, but not limited to, stainless steel or aluminum), composites (such as, but not limited to, carbon fiber composites), and polymers (such as, but not limited to, polyether ketone (PEEK), polyethylene, or ultra high molecular weight polyethylene (UHMWPE)). It may be desirable to make the working cannula from a radiolucent material such as polyetherether ketone (PEEK) to enhance visualization of the surgical site when using fluoroscopic or other imaging techniques. Further, the distal ends of the bullet-shaped dilator and tube bodies may have friction-reducing coatings such as, but not limited to, Teflon to ease insertion of the tubes into the expanded tissue. Alternatively, the dilator tubes may be polished to reduce friction. The dilator tubes may further be provided with a glare-reducing coating to minimize the reflection of light.

The metal dilator tubes, trocar and handles may be configured to be sterilized. Where elements of the system are fabricated from non-metallic materials, such elements may be disposable after use. Thus, a partially or completely disposable sequential dilation system may be provided.

The proximal ends of the dilator tubes also may have coatings, ridges, roughenings or other surface profilings to allow a surgeon to more easily grasp the dilator tubes for insertion and/or removal. In addition to the color-coded grooves mentioned above, the tubes themselves may be color-coded for easy identification of diameter, length, material, etc.

In use, the illustrated series or system of six dilator tubes may be provided with a set of two handles, with each handle configured to accept up to three dilator tubes. In one embodiment, the first handle 500 may accept dilator tubes 500, 600 and 700, while the second handle (not shown) may accept dilator tubes 800, 900 and 1000. As previously described, the bullet-shaped dilator 400 may be used to provide an initial expansion of the incision 100, and may thereafter be removed from the patient to allow the individual tubular dilator elements to be inserted to provide subsequent increased expansion of the incision. The surgeon may then engage the proximal end of the smallest tubular dilator element 600 in the appropriate handle, pressing the element into the handle 500 until the corresponding ball-detent 522 clicks into the groove 622 in the dilator element 600. The surgeon may then insert the dilator element 600 over the guide wire 200 and into the incision 100, using the handle to press the dilator into the incision against attendant tissue forces. The surgeon may also use the handle to impart a twisting or rocking motion to the dilator element to help overcome any tissue forces (frictional or otherwise) that may act on the dilator element. Once dilator tube 600 has been fully inserted into the incision 100, the handle 500 may be removed from the tube 600 by grasping the tube and pulling up on the handle 500. The axial force applied should be sufficient to overcome the spring force associated with the engaged ball detent 522, causing the ball to move into the recess in the handle, thus releasing the handle from the tube 600. The next larger dilator tube 700 may then be inserted into associated counterbore 530 of the handle 500 until the associated ball detent 532 engages groove 722 in proximal end 720 of dilator tube 700. Distal end 710 of dilator tube 700 is then placed over dilator tube 600 and pressed into incision 100, further expanding the incision 100. When dilator 700 is inserted to the proper depth, ball detent 522 may engage associated groove 622 of dilator tube 600, thus locking dilator tube 600 to the handle 500. In this condition, the handle may be locked to both dilator tubes 600, 700. Thereafter, the proximal end of dilator tube 700 may be grasped by the surgeon to maintain it in place within the patient's body while pulling up on the handle 500. This axial force may cause the ball detent 532 to disengage from groove 722 of dilator tube 700, thus detaching tube 700 from handle 500. Since the handle 500 and dilator tube 600 remain fixed together, pulling up on handle 500 also causes tube 600 to be removed from the patient. A subsequent dilator tube 800 or tubes 800-1100 may be placed and removed in sequence, as described above, until the desired expansion of the incision 100 has been achieved. The sequential installation and removal technique described herein may apply regardless of what engagement arrangement is used between the handle and dilator tubes. The only differences may be in the manner in which tube/handle engagement and disengagement is performed (e.g. using the spring pin engagement arrangement may require less force to be applied to disengage the tube and handle as compared to the ball-detent arrangement).

The number and size of dilator tubes used for a particular procedure may be based on the cross-section of incision needed for insertion of surgical instrumentation and/or for the particular procedure being performed. The outer diameters of the dilator tubes may range from about 10 mm to about 30 mm, and the increments of increase between successive dilator tubes may be from between about 1 mm to about 5 mm. Where a series of dilator tubes is used, the number of tubes provided may vary as appropriate, and the incremental increase in diameter from one tube to the next may also be varied, as long as a gradual increase in the cross-section of the incision is provided. Incremental sizing of the tubes gradually and gently increases the size of the incision, minimizing tissue tearing or other damage. In one embodiment, the increase in outside diameters between successive dilator tubes is about 2 mm. Further, depending on the number of dilator tubes required, two or more handles may be provided to accommodate the full range of diameters of dilator tubes.

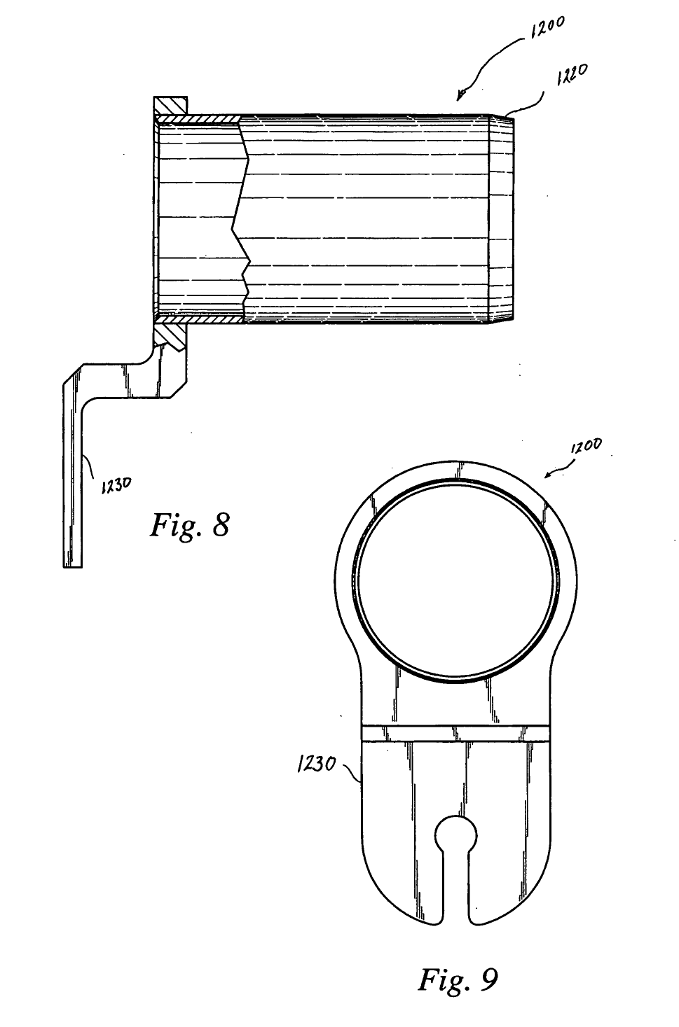

The last step in dilation may comprise inserting a working cannula 1200 (shown in FIG. 8) over the last tubular dilator element in the series. The ultimate surgical procedure may be performed through this working cannula 1200, and thus it may have an outer diameter greater than the largest dilator element in the series. The working cannula 1200 may be used to provide additional dilation of the surgical incision compared to the last-placed dilator tube in the series, and thus working cannula 1200 may have a tapered distal end 1220 to facilitate its insertion into the incision 100.

The working cannula 1200 also may have a tab or handle 1230 attached to or integral with the proximal end of the cannula. This tab or handle 1230 may be used to attach the cannula 1200 to a rigid frame to secure the position of the cannula during the remainder of the surgical procedure. Such a rigid frame may be used to secure multiple additional cannulas such as may be required for complex surgical procedures involving more than one incision (e.g. spinal fixation procedures involving the insertion of multiple pedicle screws, spinal fixation rods, inter-vertebral implants, etc.). The outside diameter of working cannula 1200 may be in a range from about 15 mm to about 100 mm.

The elements of the surgical dilator system may be provided in the form of a kit for surgical use. The kit may include at least one guide wire, a T-handle trocar, a mallet for tamping the guide wire into bone, a bullet-shaped dilator, a series of dilator tubes having different lengths and diameters as previously described, at least one tubular dilator handle, and at least one working cannula. The dilator tubes may be provided in any appropriate combination of sizes appropriate for a particular surgical use (e.g., a smaller system may be provided for pediatric use). The handles, bullet-shaped dilator, and dilator tubes may be provided in any one or combination of the materials previously identified, and may have any one or combination of friction-reducing and glare-reducing coatings or polishing. Furthermore, the dilator tubes may be color-coded for easy identification of diameter, length, material, etc.

Further, it should be understood that variations and modifications within the spirit and scope of the invention may occur to those skilled in the art to which the invention pertains. Accordingly, all expedient modifications readily attainable by one versed in the art from the disclosure set forth herein that are within the scope and spirit of the present invention are to be included as further embodiments of the present invention. The scope of the present invention is accordingly defined as set forth in the appended claims.

What is claimed is:

1. A surgical instrument comprising:
at least a first dilator element having a tissue engaging portion, a gripping element engaging portion and a length,
a gripping element comprising a gripping portion and a dilator element engaging portion, the gripping element removably engageable with the dilator element;
wherein when the gripping element and the dilator elements are engaged, the gripping element is operable for engaging the dilator element with a surgical opening.

2. The surgical instrument of claim 1, wherein the dilator element engaging portion comprises a releasable coupling configured to releasably engage the gripping element engaging portion.

3. The surgical instrument of claim 2, wherein the releasable coupling comprises at least one ball-detent mechanism and the gripping element engaging portion of the dilator element comprises a recess configured to accept at least a portion of the ball-detent mechanism, wherein engaging the ball-detent mechanism with the recess axially locks the gripping element to the first dilator element.

4. The surgical instrument of claim 3, wherein when the ball-detent mechanism of the gripping element is engaged with the recess of the first dilator element, the ball-detent mechanism may thereafter be disengaged from the recess by the application of an axial force applied in a direction tending to separate the gripping and dilator elements.

5. The surgical instrument of claim 2, wherein the releasable coupling comprises at least one spring-loaded button.

6. The surgical instrument of claim 2, wherein the gripping element comprising a wall disposed between the gripping surface and the dilator element engaging surface, the element further comprising at least one window disposed in the wall to allow visualization of at least a portion of the first dilator element within the gripping element when the dilator and gripping elements are engaged

7. The surgical instrument of claim 1, further comprising a second dilator element having a gripping element engaging portion, the gripping element comprising a second dilator element engaging surface, wherein the gripping element and the second dilator element are removably engageable with each other.

8. The surgical instrument of claim 7, the first dilator element further having first inner and outer dimensions, the second dilator element further having second inner and outer dimensions, wherein the second inner dimension is greater than the first outer dimension so that at least a portion of the second dilator element may be telescopically received within the first dilator element.

9. The surgical instrument of claim 8, the dilator engaging portion of the gripping element further configured to engage the first and second dilator elements concurrently.

10. The surgical instrument of claim 7, wherein the gripping element is selectively engageable and disengageable with the first and second dilator elements.

11. The surgical instrument of claim 10, wherein at least a portion of the first dilator element comprises a first color and at least a portion of the second dilator element comprises a second color, the first and second colors being different to provide a visual indication of dilator size.

12. The method of claim 1, wherein the gripping element has a rotational retention feature for rotationally fixing the dilator element to the gripping element.

13. The surgical instrument of claim 12, wherein the rotational retention feature comprises a keyed surface configured to engage a corresponding surface of the dilator element.

14. The surgical instrument of claim 13, wherein at least a portion of the dilator element engaging surface of the gripping element is cylindrical.

15. The surgical instrument of claim 1, wherein the tissue engaging portion of the dilator element comprises a friction reducing coating.

16. The surgical instrument of claim 1, wherein the gripping element engaging portion of the dilator element has a grip enhancing configuration.

17. The surgical instrument of claim 16, wherein the grip enhancing configuration comprises ridges, grooves, roughenings, coatings or other surface profilings.

18. A surgical dilator system comprising
at least a first dilator tube comprising an tissue engaging portion and handle engaging portion, the handle engaging portion further comprising an outer surface having a first surface feature;
a handle portion comprising:
an outer surface configured for gripping by a user, and
an inner surface configured to engage the handle engaging portion of the first dilator tube, the inner surface having a first surface feature;
wherein when the dilator tube is received within the handle the corresponding surface features provisionally axially lock the tube to the handle to allow the dilator to be inserted into a surgical opening by a user gripping the handle.

19. The surgical dilator system of claim 18, wherein the surface feature on the handle comprises at least one projection and the surface feature on the dilator tube comprises a corresponding recess.

20. The surgical dilator system of claim 18, wherein the surface feature on the handle comprises a ball element of a ball detent mechanism and the surface feature on the dilator tube comprises a corresponding recess.

21. The surgical dilator system of claim 20, the handle further comprising a first wall disposed between the inner and outer surfaces, wherein the ball detent mechanism is disposed in at least a portion of the wall.

22. The surgical dilator system of claim 18, further comprising a second dilator tube having a second handle engaging portion comprising an outer surface having a second surface feature, the handle inner surface further configured to engage the handle engaging portion of the second dilator element, the handle inner surface further comprising a second surface feature, wherein when the second dilator tube is received within the handle the corresponding surface features provisionally axially lock the second dilator tube to the handle to allow the second dilator tube to be inserted into a surgical opening by a user gripping the handle.

23. The surgical dilator system of claim 22, the first dilator tube further having first inner and outer dimensions, the second dilator tube further having second inner and outer dimensions, wherein the second inner dimension is greater than the first outer dimension so that at least a portion of the second dilator tube may be telescopically received within the first dilator tube.

24. The surgical dilator system of claim 23, the inner surface of the handle further configured to engage the first and second dilator tubes concurrently.

25. The surgical dilator system of claim 24, wherein the handle is selectively engageable and disengageable with the first and second dilator tubes.

26. The surgical dilator system of claim 18, the handle further comprising first and second concentric bores, the first bore configured to receive the first dilator tube, the second bore configured to receive the second dilator tube, the first and second bores comprising at least one surface feature configured to releasably engage respective corresponding surface features on the first and second dilator tubes.

27. The surgical dilator system of claim 26, the first dilator tube further having a first radial dimension, the second dilator tube further having a second radial dimension, wherein the first radial dimension is smaller than the second radial dimension.

28. The surgical dilator system of claim 27, the first dilator tube further having a first length, the second dilator tube having a second length, the first length greater than the second length.

29. The surgical dilator system of claim 26, the handle further comprising a first wall disposed between the outer surface and the first bore, a first window disposed within the first wall and configured to allow observation of at least a portion of the first dilator tube when the tube is positioned within the handle.

30. The surgical dilator system of claim 29, the handle further comprising a second wall disposed between the outer surface and the second bore, a second window disposed within the second wall and configured to allow observation of at least a portion of the second dilator tube when the second tube is positioned within the handle.

31. The handle of claim 26, the first and second bores each comprising a rotational locking surface configured to engage a corresponding respective locking surface of the first and second dilator tubes to rotationally lock the handle to the tubes.

32. The handle of claim 31, wherein the rotational locking surface of the first bore comprises at least one flat portion configured to engage a corresponding flat portion of the first dilator tube.

33. A method for enlarging an incision in a patient, said method comprising:
(a) making an incision through at least the skin of the patient;
(b) inserting a guide wire into the incision and advancing a distal end of the guide wire to a location adjacent a targeted surgical site in the patient's body;
(c) providing at least a first dilator element having proximal and distal ends and inner and outer surfaces;
(d) providing a first handle element configured to releasably engaging the proximal end of the dilator element;
(e) engaging the dilator element with the handle element;
(f) using the handle to position the distal end of the dilator element over the guide wire;
(g) using the handle to advance the dilator element along the guide wire until the distal end of the dilator element is positioned adjacent the surgical site; and
(h) releasing the handle element from the proximal end of the dilator element, leaving the dilator element in the incision.

34. The method of claim 33, further comprising the steps of:
(i) providing a second dilator element having proximal and distal ends and inner and outer surfaces;
(j) releasably engaging the handle element with the proximal end of the second dilator element;
(k) using the handle to position the distal end of the second dilator element over the proximal end of the first dilator element;
(l) using the handle to advance the second dilator element along the first dilator element until the distal end of the second dilator element is positioned adjacent the surgical site;
(m) engaging the handle with the proximal end of the first dilator element;
(l) releasing the handle from the second dilator element and removing first dilator element from the incision using the handle.

35. The method of claim 33, further comprising, between steps (b) and (c), the steps of: positioning a bullet-shaped dilator element over the guide wire; urging the bullet-shaped dilator through the incision; advancing the bullet-shaped dilator along the guide wire until a distal end of the dilator is positioned adjacent the surgical site; and removing the bullet-shaped dilator from the incision.

36. The method of claim 35, wherein the bullet-shaped dilator and the first dilator element are urged through the incision and advanced along the guidewire together.

37. The method of claim 33, wherein the handle has an axial retention feature for selectively axially locking the dilator elements to the handle.

38. The method of claim 37, wherein the axial retention feature comprises a ball detent mechanism disposed in the handle.

39. The method of claim 33, wherein the handle has a rotational retention feature for rotationally locking the dilator element to the handle.

40. The method of claim 33, wherein the handle comprises at least one window to allow visualization of the portion of at least one of the dilator elements within the handle.

41. The method of claim 33, wherein at least one tube has at least one window to allow at least a portion of the at least one of the dilator elements to be observed from outside the handle.

42. The method of claim 41, wherein at least one tube has a depth marking adjacent the viewing window to indicate when an associated dilator tube is fully engaged with the dilator handle.

43. The method of claim 33, further comprising the steps of: inserting a working cannula over the at least one dilator element, advancing the working cannula through the incision until the distal end of the working cannula is located adjacent the surgical site; removing the dilator element from the incision; and performing a surgical procedure at the surgical site through the working cannula.

44. A kit comprising a plurality of dilator elements each having proximal and distal ends, and at least one handle configured to removably engage the proximal ends of the plurality of dilator elements.

45. The kit of claim 44, further comprising a bullet-shaped dilator.

46. The kit of claim 44, wherein the handle has an axial retention feature for selectively axially locking each dilator element to the handle.

47. The kit of claim 44, wherein the handle has a rotational retention feature for rotationally fixing the dilator elements to the handle.

48. The kit of claim 44, further comprising a working cannula.

49. The kit of claim 44, wherein at least first and second dilator elements each have inner and outer surface dimensions, the outer surface dimension of first dilator element being smaller than the inner surface dimension of the second dilator element.

50. The kit of claim 49, wherein the first and second dilator elements each have a length, the length of the first dilator element being greater than the length of the second dilator element.

51. The sequential dilator kit of claim 44, wherein the removable handle has a ball detent mechanism to engage at least one of the dilator elements.

52. The sequential dilator kit of claim 44, wherein the removable handle includes a spring-loaded two position button to allow engagement and disengagement of the handle with at least one dilator element.

53. The sequential dilator kit of claim 44, wherein the removable handle includes a window for viewing at least a portion of one of the dilator elements within handle.

54. The sequential dilator kit of claim 44, wherein the removable handle has at least one counterbore to allow the removable handle to slidingly receive at least one dilator element.

55. The sequential dilator kit of claim 44, wherein the counterbore has a flattened side configured to mate with a corresponding surface of at least one dilator element to prevent rotational movement of the removable handle and dilator element with respect to each other.

56. The sequential dilator kit of claim 44, wherein the dilator elements are color coded.

57. The sequential dilator kit of claim 44, wherein the distal end of at least one dilator element is tapered.

58. The sequential dilator kit of claim 44, wherein the distal end of at least one dilator element has a friction-reducing coating.

59. The sequential dilator kit of claim 44, wherein the proximal end of at least one dilator element has coatings, ridges, roughening, or other surface profiling.
